(19) United States
(12) Patent Application Publication
(10) Pub. No.: US 2011/0189714 A1
(43) Pub. Date: Aug. 4, 2011

(54) MICROFLUIDIC CELL SORTER AND METHOD

(76) Inventors: Harold E. Ayliffe, Woodinville, WA (US); Curtis S. King, Kirkland, WA (US)

(21) Appl. No.: 12/699,745

(22) Filed: Feb. 3, 2010

(51) Int. Cl. C12Q 1/02; C12M 1/34

(52) U.S. Cl. 435/29; 435/287.1

(57) ABSTRACT

A biological particle sorting device and method of its use. The device includes structure arranged to urge biological particles into substantially single file travel through an interrogation zone. Operable alignment structure nonexclusively include sheathed fluid flow, capillary tubes, an orifice, and fluid microchannels. One or more detector, selected from a plurality of operable such structures, may be employed to sense the presence of a biologic particle in the interrogation zone. Certain exemplary detectors may operate on the Coulter principle, or may detect a Stokes' shift, or side-scatter radiation. Discrimination structure is generally provided to categorize particles as being in one or another sub-population of a mix of biological particles that may be carried in a fluid sample, such as by cell type, size, or the like. Killing structure, such as a laser, is disposed to neutralize all particles in any undesired sub-population while leaving undamaged the desired sub-population(s). The device may be operated to essentially purify (in a living or viable sense) a sample including biological particles that are carried in a fluid diluent.

### BACKGROUND

1. Field of the Invention

This invention relates to biological cell sorting and purification systems. Certain embodiments are particularly adapted for use in microfluidic plumbing arrangements to selectively kill one or more entire population of undesired cells.

2. State of the Art

It is sometimes desirable to sort one or more selected population of biological particles from a sample containing a plurality of different populations of particles. For example, it may be desired to select for culture only a subset of particles that are present in a mixture of particles. If physical cell sorting is not done, selective cell killing may sometimes be done instead. However, commercially available killing devices and methodologies, such as lethal reagents that may be added to a fluid sample, are less flexible and precise than desired.

Conventional cell sorting devices tend to be complex, bulky, and expensive. An exemplary cell sorter based on a cytometric device with sheath flow is disclosed in U.S. utility Pat. No. 7,392,908 to Frazier. A particle analyzer including side-scatter detection and a cytometric device with capillary fluid flow is disclosed in U.S. Pat. No. 7,410,809 to Goix, et al. Causing magnetic beads to bind to selected cells is a known useful step in a technique to “hold back” and remove the bound cells from a population of cells, as disclosed in U.S. Pat. Nos. 7,417,418 and 7,579,823 to Ayliffe. The latter two utility patents also disclose microfluidic devices that are useful to interrogate biological particles as such particles flow through a thin film sensor.

It would be an improvement to provide a device, and a method of its use, for rapidly, inexpensively, and accurately purifying a viable population of biological particles by discriminately killing all of, or substantially all of, the undesired particles.

### BRIEF SUMMARY OF THE INVENTION

This invention provides an apparatus for interrogating and “purifying” a sample of fluid that carries biological particles. The purification process includes killing all, or substantially all, biological particles that do not reside in a population of desired, or at least tolerable, particles. Preferred embodiments of the invention include alignment structure, detection structure, discrimination structure, killing structure, and a trigger operable to actuate the killing structure responsive to input received from one or both of the detection structure and the discrimination structure.

A workable alignment structure is configured and arranged to urge biological particles, which are carried in a fluid, toward substantially single-file travel through an interrogation zone. Workable alignment structure comprises a fluid sheath (such as provided in cytometry devices), a capillary device, or a fluid-carrying channel, such as may be formed in a thin film layer. An interrogation zone may broadly be defined as an area or volume in which information may be gathered about particles carried in a fluid diluent. Sometimes, an interrogation zone is carried on a disposable device that is adapted for one-time-use. A currently preferred such disposable device is embodied as a microfluidic cartridge.

Detection structure may include any structure operable to detect the presence of a first biological particle in the interrogation zone. Exemplary detection structure comprises a plurality of electrodes disposed in operable association with an orifice effective to permit detecting the presence of a particle in said interrogation zone by way of the Coulter principle. Certain detection structure may also characterize one or more particle characteristic, such as particle size. Alternative detection structure comprise a radiation source disposed to impinge radiation comprising substantially a first frequency into the interrogation zone; and a radiation detector disposed to detect a Stokes' shift in the first frequency. Another alternative detection structure comprises a radiation source disposed to impinge radiation comprising substantially a first frequency into the interrogation zone; and a radiation detector disposed to detect side-scatter of the radiation.

Discrimination structure is operable to distinguish the first biological particle as either residing inside a defined population of particles, or not. Killing structure is configured and arranged substantially discriminately to kill a selected biological particle in a killing zone that is associated with the interrogation zone.

One workable trigger is adapted to operate the killing structure in the case when a detected biological particle is both present in said killing zone; and resides inside the defined population of particles. In other cases, a workable trigger is adapted to operate the killing structure in the case when a detected biological particle is both: present in the killing zone; and resides outside the defined population of particles.

A killing zone may be disposed as a sub-portion of the interrogation zone. Sometimes, a killing zone may be disposed downstream of the interrogation zone by a known time-of-flight for a biological particle to be killed. Sometimes, a killing zone may be disposed downstream of detection structure by a known time-of-flight for a biological particle to be killed.

One operable killing structure comprises a radiation source having sufficient discharged energy density to permit exposing a biological particle, during the time that biological particle is passing through the killing zone, to at least that quantity of energy sufficient to kill the biological particle. One exemplary killing structure comprises a laser. Alternative killing structure within contemplation nonexclusively includes electric elements capable of causing voltage or current spikes, LEDs, and Arc lamps of various types.

Certain embodiments of the invention may be structured to form a microfluidic device including alignment structure configured and arranged to urge biological particles, which are carried in a fluid, toward substantially single-file travel through an interrogation zone. One such device also includes detection structure operable to detect the presence of a first biological particle in the interrogation zone using electrical impedance in accordance with the Coulter principle. Further, that device includes discrimination structure operable to distinguish the first biological particle as either residing inside a defined population of particles, or not. The exemplary device also includes killing structure configured and arranged substantially discriminately to kill a selected biological particle in a killing zone that is associated with the interrogation zone. Finally, the exemplary device also includes a trigger operable to discriminately actuate the killing structure responsive to input received from both of the detection structure and the discrimination structure.

A device structured according to certain principles of the instant invention may be used in a method to identify and kill selected biological particles. The method includes providing a microfluidic device comprising: alignment structure, detection structure, discrimination structure, killing structure, and a trigger operable to actuate the killing structure responsive to input received from one or both of the detection structure and the discrimination structure. Broadly, the alignment structure should be configured and arranged to urge biological particles, which are carried in a fluid, toward substantially single-file travel through an interrogation zone. Workable detection structure includes any structure operable to detect the presence of a first biological particle in the interrogation zone. Exemplary discrimination structure is operable to distinguish the first biological particle as either residing inside a defined population of particles, or not. Operable killing structure is configured and arranged substantially discriminately to kill a selected biological particle in a killing zone that is associated with the interrogation zone. The method continues by introducing a fluid sample, comprising biological particles carried by a dilutant fluid medium, for flow of the sample past the alignment structure. Then, the method includes operating the trigger to actuate the killing structure effective to kill a sub-population of biological particles responsive to input received from one or both of the detection structure and the discrimination structure as the sample flows through the device. Sometimes, the sub-population is defined by a common characteristic that is directly detected by the discrimination structure. Other times, the sub-population is defined by a common characteristic that is not directly detected by the discrimination structure.

### BRIEF DESCRIPTION OF THE DRAWINGS

In the drawings, which illustrate what are currently considered to be the best modes for carrying out the invention:

FIG. 1 is a schematic representation of an embodiment of the instant invention in workable association with a sheath fluid system;

FIG. 2 is a schematic representation of an embodiment of the instant invention in workable association with a capillary tube based flow system;

FIG. 3 is a schematic representation of a first embodiment of the instant invention in workable association with aperture fluid flow and radiation detection;

FIG. 4 is a schematic representation of a second embodiment of the instant invention in workable association with aperture fluid flow and radiation detection;

FIG. 5 is a cross-section view in elevation of an embodiment of the instant invention including elements arranged to permit electrical property interrogation and radiation detection;

FIG. 6 is a cross-section view in elevation of an embodiment of the instant invention including elements arranged to permit side-scatter and Stokes' shift radiation detection;

FIG. 7 is a plan view of a portion of the assembly illustrated in FIG. 6;

FIG. 8 is an exploded assembly view in perspective from above of a workable microfluidic device including constituent layers of thin film and including elements arranged to permit electrical property interrogation and radiation detection;

FIG. 9 is a top plan view of the assembly illustrated in FIG. 8;

FIG. 10 is a representative plot of measured electrical property vs. time; and

FIG. 11 is a representative plot of measured intensity vs. wavelength.

### DETAILED DESCRIPTION OF THE ILLUSTRATED EMBODIMENTS

Reference will now be made to the drawings in which the various elements of the illustrated embodiments will be given numerical designations and in which the invention will be discussed so as to enable one skilled in the art to make and use the invention. It is to be understood that the following description is only exemplary of the principles of the present invention, and should not be viewed as narrowing the claims which follow.

Currently preferred embodiments of the present invention provide low-cost, disposable, sensors operable to perform analyses of various sorts on particles that are carried in a fluid. Sensors structured according to certain principles of the instant invention may be used once, and discarded. However, it is within contemplation that such sensors may alternatively be reused a number of times.

Examples of analyses in which embodiments of the invention may be used to advantage include, without limitation, counting, characterizing, or detecting members of any cultured cells, and in particular blood cell analyses such as counting red blood cells (RBCs) and/or white blood cells (WBCs), complete blood counts (CBCs), CD4/CD8 white blood cell counting for HIV+ individuals; whole milk analysis; sperm count in semen samples; and generally those analyses involving numerical evaluation or particle size distribution for a particle-bearing fluid (including nonbiolgical). Embodiments of the invention may be used to provide rapid and point-of-care testing, including home market blood diagnostic tests. Certain embodiments may be used as an automated laboratory research cell counter to replace manual hemacytometry. Preferred embodiments of the invention are adapted to provide a low-cost fluorescence activated cell sorter (FACS) that may be used to selectively kill biological particles and thereby “purify” a fluid sample.

For convenience in this disclosure, the invention will generally be described with reference to its use as a particle detector and killer. Such description is not intended to limit the scope of the instant invention in any way. It is recognized that certain embodiments of the invention may be used simply to detect passage of particles, e.g. for counting. Other embodiments may be structured to determine particle characteristics, such as size, or type, thereby permitting discrimination analyses. Furthermore, for convenience, the term “fluid” may be used herein to encompass a fluid mix including a fluid base formed by one or more diluents and particles of one or more types suspended or otherwise distributed in that fluid base. Particles are assumed to have a characteristic “size”, which may sometimes be referred to as a diameter, for convenience. Currently preferred embodiments of the invention are adapted to interrogate particles found in whole blood samples, and this disclosure is structured accordingly. However, such is not intended to limit, in any way, the application of the invention to other fluids including fluids with particles having larger or smaller sizes, as compared to blood cells.

In this disclosure, “single-file travel” is defined different than literally according to a dictionary definition. For purpose of this disclosure, substantially single-file travel may be defined as an arrangement of particles sufficiently spread apart and sequentially organized as to permit reasonably accurate detection and discriminate killing of particles of interest. When two particles are in the interrogation zone at the same, it is called coincidence, and there are ways to mathematically correct for it. Calibration may be performed using solutions having a known particle density (e.g. solutions of latex beads having a characteristic size similar to particle(s) of interest). Also, dilution of the particles in a fluid carrier may contribute to organizing particle travel. As a non-limiting example, the desired particle density to urge single-file travel and reduce or avoid coincidence is approximately between about 3×10³ to about 3×10⁵ cells/ml, where the particle size is on the order of the size of a red blood cell.

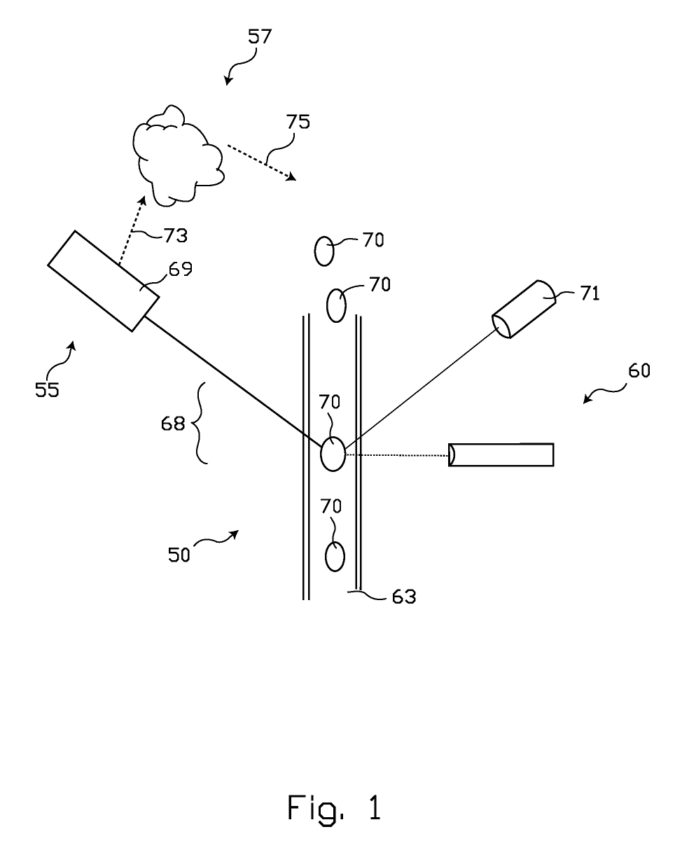

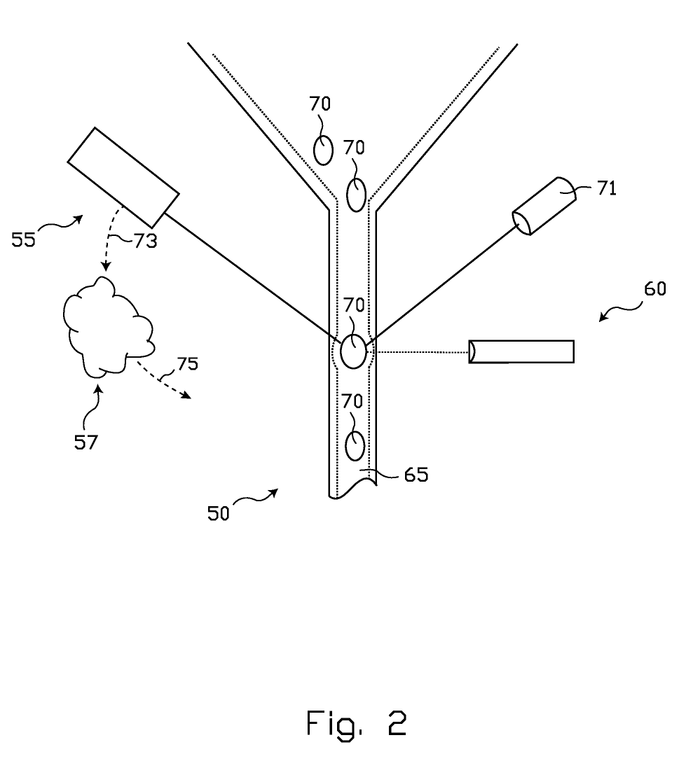

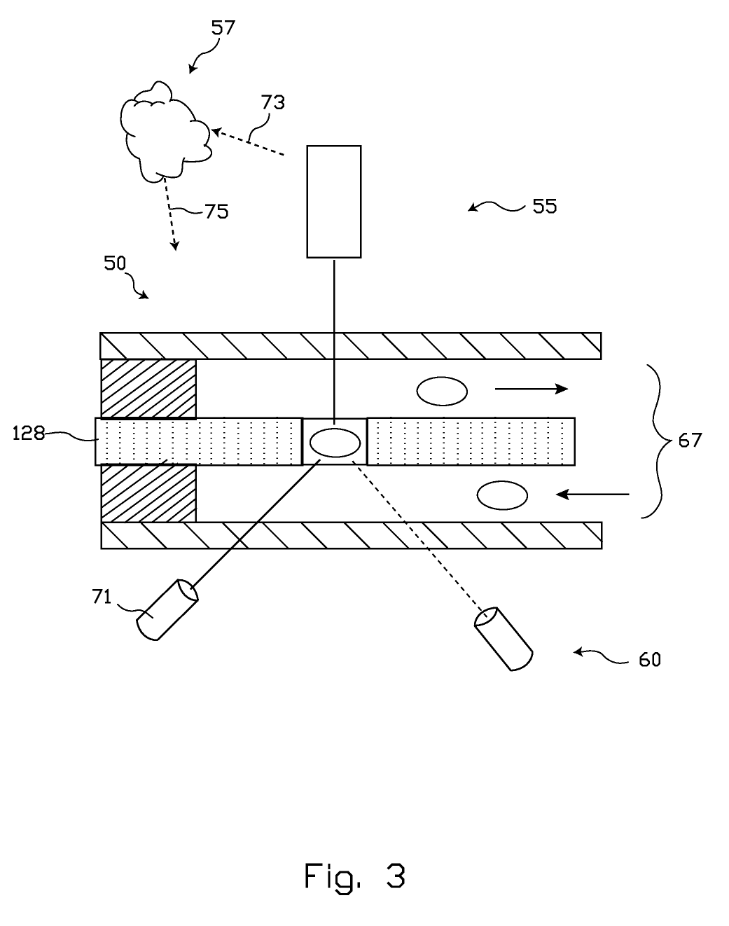

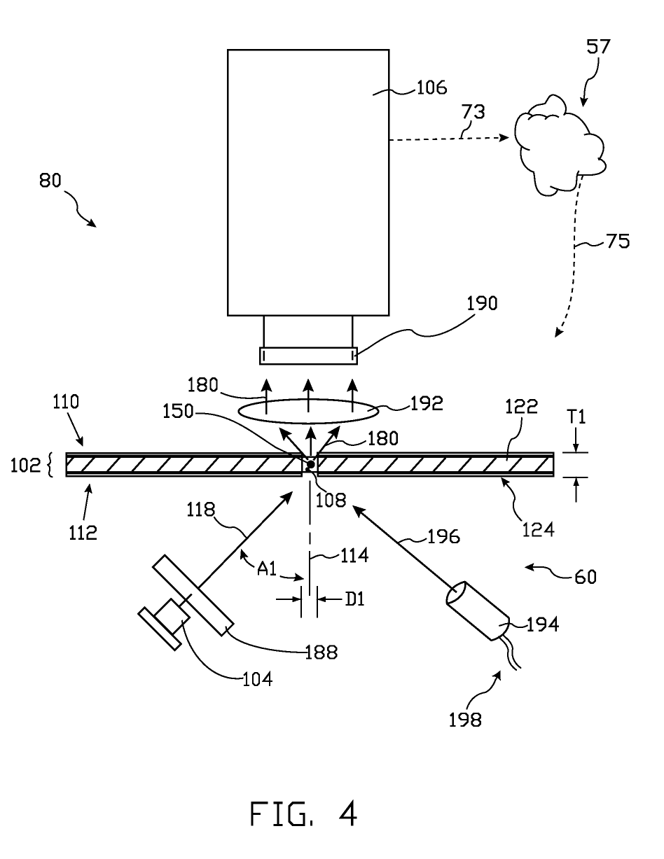

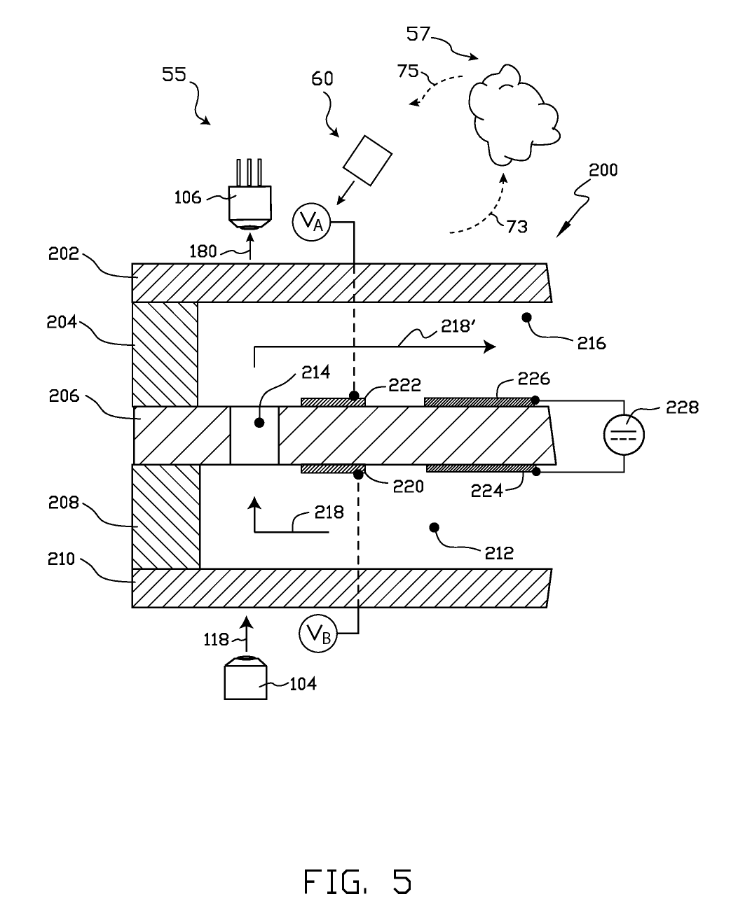

As illustrated in FIGS. 1-3, operable embodiments structured according to certain aspects of the invention include alignment structure, generally 50, detection structure, generally 55, discrimination structure, generally 57, and killing structure, generally 60. In general, alignment structure 50 is effective to urge transit of particles of interest (e.g. biological cells) into substantially single-file for travel of those particles through an interrogation zone. Workable alignment structure 50 nonexclusively includes the sheath fluid system 63 in FIG. 1; the capillary fluid system 65 in FIG. 2; and the thin film channel system 67 in FIG. 3.

Detection structure 55 encompasses any device, or assembly of devices and elements, operable to detect the presence of a biological particle in an interrogation zone 68. Broadly, an interrogation zone 68 is an area in which information about a particle may be determined. Exemplary such information includes particle size, type, and presence. Desirably, alignment structure 50 cooperates with an amount of sample dilution to reduce particle coincidence and urge particles into single-file travel through the interrogation zone 68.

In FIG. 1, detection structure 55 includes a radiation detector 69, and a cooperating source of radiation 71 that is positioned to impinge into the interrogation zone. Workable sources of radiation include lamps, LEDs, and lasers, for non-limiting examples. In one embodiment, one or more radiation detector 69 may be configured and arranged to detect side-scatter radiation from particles, such as biological cells 70, which are traveling through the interrogation zone 68. Alternatively, or additionally, a radiation detector 69 may be configured and arranged to detect radiation emitted by a particle undergoing a Stokes' shift fluorescence phenomena in the interrogation zone 68.

Discrimination structure 57 encompasses any device, or assembly of devices and elements, operable to distinguish biological particles as either residing inside a defined population of particles, or not. In FIG. 1, discrimination structure 57 may encompass electrical circuitry and components, one or more microprocessor, computer memory, data structures and tables and/or threshold values stored in the memory, and software that may be variously programmed to operate the apparatus. The discrimination structure 57 in FIG. 1 receives feedback, or data input indicated at 73, from one or more detector 69. In an exemplary case, a signal received by detection structure 55 due to side-scatter radiation may be employed to indicate presence of a particle in the interrogation zone 68. Detection of Stoke's shift fluorescence may be further employed to determine if the particle is, or is not, in a particular population of particles. Broadly, particles may be sorted into various populations based upon any detectable characteristic, including electrical property, radiological property, particle size, and the like.

Killing structure 60 encompasses any device, or assembly of devices and elements, configured and arranged substantially discriminately to kill a selected biological particle in a killing zone that is associated with the interrogation zone. Operable killing structure 60 nonexclusively includes lasers and other energy-outputting devices. Although it is not required, typically a dedicated killing structure 60, such as a laser, is selected having a significantly different wavelength compared to the excitation radiation source 71. For example, a killing laser is typically selected to emit in the ultraviolet (UV) spectrum, or infrared (IR) spectrum. In contrast, an excitation radiation source 71 typically emits radiation in the visible spectrum. However, it is within contemplation that the intensity of the excitation source 71 could simply be increased sufficiently to effect a kill when desired.

Assemblies structured according to certain principles of the invention also include a trigger operable to actuate a killing structure 60 responsive to analysis of data received from one or both of a detection structure 55 and a discrimination structure 57. With reference still to FIG. 1, trigger 75 causes the killing structure 60 to operate effective to kill one or more selected biological particle. An operable trigger 75 may include structure associated with detection structure 55 and discrimination structure 57. Software may be provided as a portion of a programmable trigger 75 to actuate a killing structure 60 in certain desired instances, and not in other instances.

For example, and with further reference to FIG. 1, a particle may detected in the interrogation zone 68 by a detection structure 55 that detects side-scatter radiation. Further, the particle may be emitting Stokes' shift fluorescence as a result of a fluorescing marker bound to the cell and indicating the cell is definitely in a certain population of cells. In the case where that population of cells is desired to be killed to “purify” the sample, trigger 75 may cause the killing structure 60 to emit a lethal dose of radiation effective to kill that cell, then to terminate killing operation while subsequent desirable particles flow through the interrogation zone. In the reverse scenario, tagged or bound particles may constitute the population of desired particles, and all detected and untagged particles may be killed.

With reference now to FIG. 4, an arrangement of structures illustrating certain principles of operation of the invention is indicated generally at 80. As illustrated, embodiment 80 includes an opaque member, generally indicated at 102, disposed between a radiation source 104 and a radiation detector 106. Opaque member 102 is provided as a portion of structure arranged to cause a desired fluid flow of a fluid sample including biological particles of interest. Sometimes, opaque member 102 may be made reference to as an interrogation layer, because layer 102 is associated with an interrogation zone. At least one orifice 108 is disposed in opaque member 102 to provide a flow path between a first side, generally indicated at 110, and a second side, generally indicated at 112. Orifice 108 may be characterized as having a through-axis 114 along which fluid may flow between the first and second sides 110 and 112 of opaque member 102, respectively.

The thickness, T1, of an opaque member and characteristic size, D1, of an orifice 108 are typically sized in agreement with a size of a particle of interest to promote single-file travel of the particle through the opaque member, and to have substantially only one particle inside the orifice at a time. In the case where the apparatus is used to interrogate blood cells, the thickness of the opaque member may typically range between about 10 microns and about 300 microns, with a thickness of about 125 microns being currently preferred. The diameter, or other characteristic size of the orifice, may range between about 2 and 200 microns, with a diameter of about 50 microns being currently preferred.

An operable opaque member 102 may function, in part, to reduce the quantity of primary radiation 118 (or sometimes characterized as excitation radiation) that is emitted by source 104, which is received and detected by radiation detector 106. Primary radiation 118 is illustrated as a vector having a direction. Desirably, substantially all of the primary radiation 118 is prevented from being detected by the radiation detector 106. In any case, operable embodiments are structured to resist saturation of the detector 106 by primary radiation 118. In certain embodiments, primary radiation 118 may simply pass through orifice 108 for reception by the radiation detector 106. Therefore, as will be further detailed below, certain embodiments may employ one or more selective radiation filters as a measure to control radiation received by detector 106, or alternatively, direct primary radiation 118 at an angle with respect to the detector 106.

The opaque member 102 illustrated in FIG. 4 includes a core element 122, carrying a first coating 124 disposed on first side 110, and a second coating 126 disposed on second side 112. An alternative core element may be formed from a core element having a coating on a single side. The illustrated coatings 124, 126 cooperatively form a barrier to transmission of excitation radiation through the core element 122. Of course, it is also within contemplation to alternatively use a bare core element that is, itself, inherently resistant to transmission of radiation (e.g. opaque core 128 in FIG. 3). One currently preferred core includes opaque polyamide film that transmits very little light through the film, so no metallizing, or other barrier element, is required. However, certain embodiments may even have an interrogation layer 102 that is substantially transparent to primary radiation 118.

A workable core 122 for use in detecting small sized particles can be formed from a thin polymer film, such as PET having a thickness of about 0.005 inches. Such polymer material is substantially permeable to radiation, so one or more coatings, such as either or both of coating 124 and 126, can be applied to such core material, if desired. A workable coating includes a metal or alloy of metals that can be applied as a thin layer, such as by sputtering, vapor deposition, or other well-known technique. Ideally, such a layer should be at least about 2-times as thick as the wavelength of the primary radiation, e.g. about 1 μm in one operable embodiment. The resulting metallized film may be essentially impervious to transmission of radiation, except where interrupted by an orifice. Aluminum is one metal suitable for application on a core 122 as a coating 124 and/or 126.

The apparatus 80 illustrated in FIG. 4 is configured to urge a plurality of particles 150 in substantially single-file through orifice 108. A particle 150 typically passes through an excitation zone as the particle approaches, passes through, and departs from the orifice 108. Of note, the direction of particle-bearing fluid flow may be in either direction through orifice 108. An excitation zone typically includes the through-channel defined by orifice 108. An excitation zone may also include a volume disposed “above” and or a volume disposed “below” the orifice 108, which encompass a volume in which a particle may reside and be in contact with primary radiation. In the excitation zone, primary radiation 108 impinged upon particles causes certain particles to fluoresce (undergo a Stokes-shift), thereby emitting radiation at a different wavelength compared to the primary radiation 108 and in substantially all three ordinate directions. The fluorescence radiation emitted by those certain particles is then detected by the radiation detector 106.

It should be noted, for purpose of this disclosure, that the term “wavelength” is typically employed not necessarily with reference only to a single specific wavelength, but rather may encompass a spread of wavelengths grouped about a characteristic, or representative, wavelength. With reference to FIG. 11, the characteristic wavelength F1 (e.g. excitation wavelength) of the primary radiation 118 is sufficiently different from the characteristic wavelength F2 of the fluorescence (e.g. emission wavelength) to enable differentiation between the two. Furthermore, the difference between such characteristic wavelengths, or Stokes-shift differential, is desirably sufficiently different to enable, in certain embodiments, including a selective-pass filter element between the radiation source 104 and detector 106 effective to block transmission of primary radiation 118 toward the detector 106, while permitting transmission of the fluorescence through the selective-pass filter to the detector 106.

With reference still to FIG. 4, the opaque member 102 in embodiment 80 may essentially be disposed in a suitably sized container that is divided into two portions by the opaque member. Flow of fluid (and particles entrained in that fluid) through the orifice 108 could be controlled by a difference in pressure between the two divided portions. However, it is typically desired to provide more control over the flow path of particles in the vicinity of the orifice 108 than such an embodiment would permit. For example, a clump of particles disposed near an entrance or exit of the orifice 108 could shield a particle of interest from the primary radiation 118 to the extent that fluorescence does not occur, thereby causing a miscount, or preventing detection of such a shielded particle of interest. Therefore, it is preferred to provide a channel system to control flow of fluid in the vicinity of the orifice 108 and form a robust interrogation zone.

Sometimes, and as illustrated in FIG. 4, it is preferred to apply primary radiation 118 at an acute angle A1 to axis 114 of orifice 108. In such case, the opaque member 102 may even function substantially as an operable filter to resist direct transmission of primary radiation 118 to a radiation detector. As illustrated, radiation vector 118 can be oriented to pass through, or partially into, orifice 108 without being detected by radiation detector 106. However, when a tagged particle 150 is present in an excitation zone (such as orifice 108 as illustrated), the resulting fluorescence 180 may still be detected by the radiation detector 106. While a workable angle A1 may be between 0 and 90 degrees, it is currently preferred for angle A1 to be between about 15 and about 75 degrees.

A radiation source 104 may be formed from a broad spectrum radiation emitter, such as a white light source. In such case, it is typically preferred to include a pre-filter 188 adapted to pass, or transmit, radiation only in a relatively narrow band encompassing the characteristic value required to excite a particular fluorescing agent associated with a particle of interest. It is generally a good idea to limit the quantity of applied radiation 118 that is outside the excitation wavelength to reduce likelihood of undesired saturation of the radiation detector, and consequent inability to detect particles of interest.

In one embodiment adapted to interrogate blood cells, it is currently preferred to use a red diode laser, and to include a short pass filter (after the diode laser), or excitation filter, that passes primary light radiation with wavelengths shorter than about 642 nm. It is also currently preferred to include a band pass filter (prior to the photodetector) with a peak that matches a particular selected fluorescence peak. Commercially available dyes may be obtained having characteristic fluorescent peaks at 660, 694, 725, and 775 nanometers. Long pass filters are also often used in place of band-pass filters prior to the photodetector. The pipette tip “cap layer” and “substrate” can also be designed to act as optical filters to aid or eliminate the need for the traditional excitation and emission filters. In this disclosure, “Post filter” may more conventionally be referred to as an “emission filter”.

With continued reference to FIG. 4, sometimes it is preferred to include an emission filter 190 that resists transmission of radiation outside the characteristic wavelength of the fluorescence 180. Such an arrangement reduces background noise and helps to avoid false readings indicative of presence of a particle of interest in an excitation zone. Also, to assist in obtaining a strong signal, an optical enhancement, such as a lens 192, can be included to gather fluorescence 180 and direct such radiation toward the radiation detector 106. Illustrated lens 192 may be characterized as an aspheric collecting lens (or doublet), and typically is disposed to focus on a point located inside the orifice 108.

Killing structure 60, such as laser 194, is disposed to permit impinging lethal radiation 196 onto biological particles that are members of one or more undesired population. Detection of the presence of a particle can be determined by radiation detector 106, or with alternative detection structure. Information 73 from radiation detector 106 may be input to discrimination structure 57. When the particle is determined to be a member of a population that is desired to be removed to “purify” the sample, trigger 75 may enable discharge of the killing laser 194. Power for the killing laser 194 can be provided by way of wires generally indicated at 198.

It is within contemplation that a device structured according to certain principles of the instant invention may, or may not, include one or more sensor component, such as an electrode, disposed in various patterns, and at various places, for contact with the fluid flowing through a conduit in the device, e.g. for impedance-based particle detection and other interrogation. Selected operable arrangements of such interrogation structure is disclosed in U.S. patent application Ser. No. 11/800,167, titled “THIN FILM PARTICLE SENSOR, and filed on May 4, 2007, the entire contents of which are hereby incorporated as though set forth herein in its entirety.

FIG. 5 illustrates certain operational details of a currently preferred sensor component, generally indicated at 200, structured according to certain principles of the instant invention. As illustrated, sensor 200 includes a sandwich of five layers, which are respectively denoted by numerals 202, 204, 206, 208, and 210, from top-to-bottom. A first portion 212 of a conduit to carry fluid through the sensor component 200 is formed in layer 208. Portion 212 is disposed parallel to, and within, the layers. A second portion 214 of the fluid conduit passes through layer 206, and may be characterized as a tunnel. A third portion 216 of the fluid conduit is formed in layer 204. Fluid flow through the conduit is indicated by arrows 218 and 218′. Fluid flowing through the first and third portions flows in a direction generally parallel to the layers, whereas fluid flowing in the second portion flows generally perpendicular to the layers.

It is within contemplation that two or more of the illustrated layers may be concatenated, or combined. Rather than carving a channel out of a layer, a channel may be formed in a single layer by machining or etching a channel into a single layer, or by embossing, or folding the layer to include a space due to a local 3-dimensional formation of the substantially planar layer. For example, illustrated layers 202 and 204 may be combined in such manner. Similarly, illustrated layers 208 and 210 may be replaced by a single, concatenated, layer.

With continued reference to FIG. 5, middle layer 206 carries a plurality of electrodes arranged to dispose a plurality of electrodes in a 3-dimensional array in space. Sometimes, such electrodes are arranged to permit their electrical communication with electrical surface connectors disposed on a single side of the sandwich, as will be explained further below. As illustrated, fluid flow indicated by arrows 218 and 218′ passes over a pair of electrodes 220, 222, respectively. However, in alternative embodiments within contemplation, one or the other of electrodes 220, 222 may not be present. Typically, structure associated with flow portion 214 is arranged to urge particles, which are carried in a fluid medium, into substantially single-file travel through an interrogation zone associated with one of, or both of, electrodes 220, 222. Electrodes 220, 222 may sometimes be made reference to as interrogation electrodes. In certain applications, an electrical property, such as a current, voltage, resistance, or impedance indicated at V_{A} and V_{B}, may be measured between electrodes 220, 222, or between one of, or both of, such electrodes and a reference. Any of the illustrated electrodes, or alternatively structured and arranged electrodes, may be used as a portion of killing structure to apply a voltage or current spike to selected cells effective to purify a sample in real-time on a substantially cell-by-cell basis.

Certain sensor embodiments employ a stimulation signal based upon driving a desired current through an electrolytic fluid conductor. In such case, it can be advantageous to make certain fluid flow channel portions approximately as wide as possible, while still achieving complete wet-out of the stimulated electrodes. Such channel width is helpful because it allows for larger surface area of the stimulated electrodes, and lowers total circuit impedance and improves signal to noise ratios. Exemplary embodiments used to interrogate blood samples include channel portions that are about 0.10″ wide and about 0.003″ high in the vicinity of the stimulated electrodes.

One design consideration concerns wettability of the electrodes. At some aspect ratio of channel height to width, the electrodes MAY not fully wet in some areas, leading to unstable electrical signals and increased noise. To a certain point, higher channels help reduce impedance and improve wettability. Desirably, especially in the case of interrogation electrodes, side-to-side wetting essentially occurs by the time the fluid front reaches the second end of the electrode along the channel axis. Of course, wetting agents may also be added to a fluid sample, to achieve additional wetting capability.

Still with reference to FIG. 5, note that electrodes 220 and 222 are illustrated in an arrangement that promotes complete wet-out of each respective electrode independent of fluid flow through the tunnel forming flow portion 214. That is, in certain preferred embodiments, the entire length of an electrode is disposed either upstream or downstream of the tunnel forming flow portion 214. In such case, the “length” of the electrode is defined with respect to an axis of flow along a portion of the conduit in which the electrode resides. The result of such an arrangement is that the electrode is at least substantially fully wetted independent of tunnel flow, and will therefore provide a stable, repeatable, and high-fidelity signal with reduced noise. In contrast, an electrode having a tunnel passing through itself may provide an unstable signal as the wetted area changes over time. Also, one or more bubble may be trapped in a dead-end, or eddy-area disposed near the tunnel (essentially avoiding downstream fluid flow), thereby variably reducing the wetted surface area of a tunnel-penetrated electrode, and potentially introducing undesired noise in a data signal.

In general, disposing the electrodes 220 and 222 closer to the tunnel portion 214 is better (e.g., gives lower solution impedance contribution), but the system would also work with such electrodes being disposed fairly far away. Similarly, a stimulation signal (such as electrical current) could be delivered using alternatively structured electrodes, even such as a wire placed in the fluid channel at some distance from the interrogation zone. The current may be delivered from fairly far away, but the trade off is that at some distance, the electrically restrictive nature of the extended channel will begin to deteriorate the signal to noise ratios (as total cell sensing zone impedance increases).

With continued reference to FIG. 5, electrode 224 is disposed for contact with fluid in conduit flow portion 212. Electrode 226 is disposed for contact with fluid in flow portion 216. It is currently preferred for electrodes 224, 226 to also be carried on a surface of interrogation layer 206, although other configurations are also workable. Note that an interrogation layer, such as an alternative to illustrated single layer 206, may be made up from a plurality of sub-component layers. In general, electrodes 224, 226 are disposed on opposite sides of the interrogation zone, and may sometimes be made reference to as stimulated electrodes. In certain applications, a signal generator 228 is placed into electrical communication with electrodes 224 and 226 to input a known stimulus to the sensor 200. However, it is within contemplation for one or both of electrodes 224, 226 to not be present in alternative operable sensors structured according to certain principles of the instant invention. In alternative configurations, any electrode in the sensor 200 may be used as either a stimulated electrode or interrogation electrode.

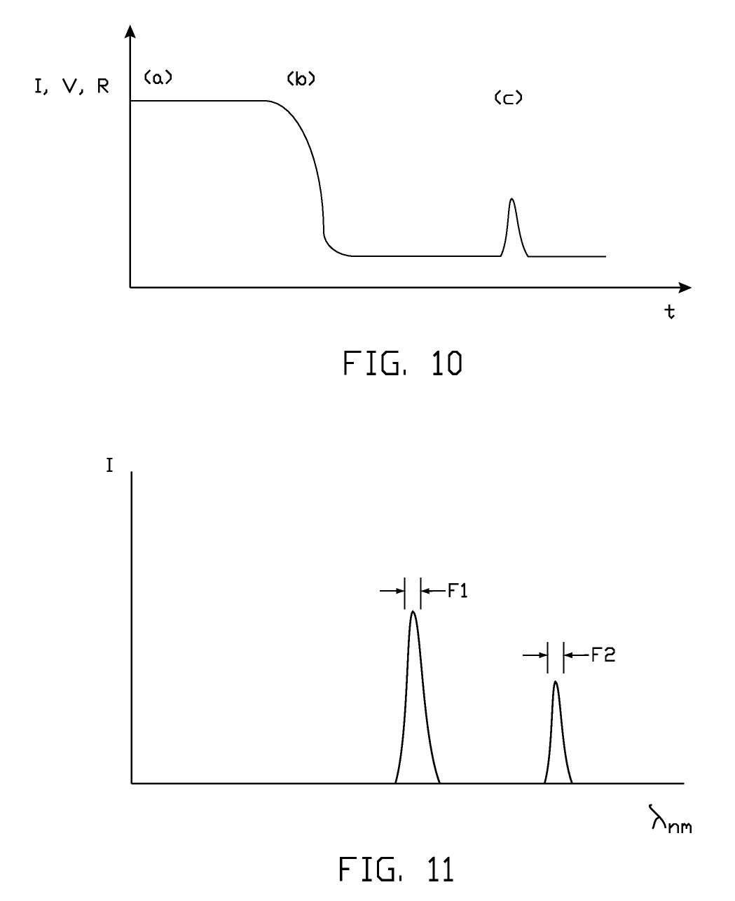

Electrodes may be positioned at a plurality of useful locations along a fluid channel. One or more electrical property may be monitored between strategically positioned electrodes to obtain information about the sample, and/or particles carried in the fluid. For example, with reference to FIG. 10, impedance measured between a pair of electrodes in a dry channel has a high value, indicated at (a). When the electrolytic diluent fluid fills and wets the channel between electrodes, the measured impedance drops, as indicated at (b). Therefore, the location of a fluid wave-front may be determined by monitoring an electrical property between strategically located electrodes. Such electrode placements may be used as event triggers, such as to start and stop data collection, and to verify absence of bubbles and processing of a desired volume disposed between electrode triggers. When a particle obstructs an interrogation aperture, a spike is measured, as indicated at (c), in accordance with the Coulter principle. Therefore, presence of particles can also be electrically determined.

As illustrated, top cap layer 202 and bottom cap layer 210 may be structured to permit application of stimulation radiation 118 into the interrogation zone associated with aperture 214. Emitted fluorescence 180 may then be detected by radiation detector 106 of detection structure 55. Presence of a cell may be detected by monitoring a radiological property such as side-scatter or fluorescence, and/or by monitoring an electrical property between a pair of electrodes, or between an electrode and a ground reference. In the event that a cell is detected in the interrogation zone, discrimination structure 57 is operable to distinguish in which population the cell resides. Discrimination structure 57 provides real-time decision making capability on a substantially cell-by-cell basis. Desirable cells are permitted to pass through the interrogation zone without incident. However, cells in undesired population(s) are desirably killed on a substantially cell-by-cell basis by killing structure 60, which is discriminately controlled by trigger 75. Actual killing of a particular cell may occur in real-time, or cell death may inevitably follow subsequent to treatment received by a cell from a killing structure 60. The resulting collected sample is therefore “purified”, in that the remaining viable cells are all members of a desired population of cells. The “purified” sample may then be manipulated or further interrogated as desired.

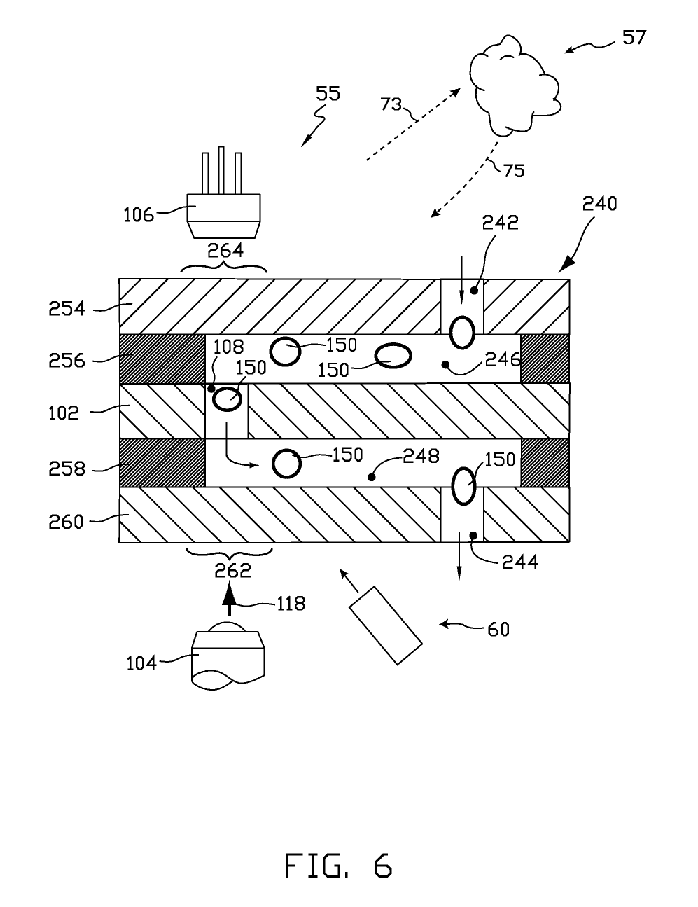

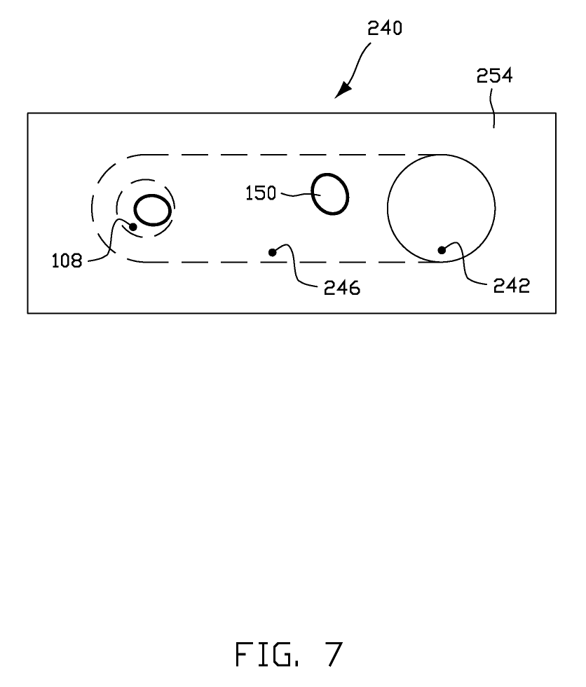

One multi-layered channel embodiment, generally indicated at 240 and illustrated in FIG. 6, provides a plumbing arrangement that is structured to resist particle clumping near the orifice 108, and consequential lack of detection of a particle of interest. Multilayer assembly 240 is structured to urge fluid flow through the orifice 108 in a direction that is essentially orthogonal to fluid flow in channel portions adjacent to, and upstream and downstream of, the orifice 108. Such fluid flow resists stacking of particles in a thickness direction of the plumbing arrangement 240, and thereby reduces likelihood of undetected particles of interest.

Plumbing arrangement 240 includes five layers configured and arranged to form a channel system effective to direct flow of particle bearing fluid from a supply chamber 242, through orifice 108 in an opaque member 102, and toward a waste chamber 244. Desirably, a depth of fluid guiding channels 246 and 248 is sized in general agreement with a size of a particle 250, to resist “stacking” particles near the orifice 108. Fluid can be moved about on the device 240 by imposing a difference in pressure between chambers 242 and 244, or across orifice 108 disposed in opaque member 102. For example, a positive pressure may be applied to the supply chamber 242. Alternatively, a negative pressure (vacuum) may be applied to the waste chamber 244. Both positive and negative pressures may be applied, in certain cases. Alternative fluid motive elements, such as one or more pumps, may be employed to control particle travel through opaque member 102.

Although both of supply chamber 242 and waste chamber 244 are illustrated as being open, it is within contemplation for one or both to be arranged to substantially contain the fluid sample within a plumbing device that includes a multilayer embodiment 240. Also of note, although a top-down fluid flow is illustrated in FIG. 6, fluid flow may be established in either direction through orifice 108. In one reverse-flow configuration, the positions of supply chamber 242 and waste chamber 244 would simply be reversed from their illustrated positions. In an alternative reverse-flow arrangement, the positions of the radiation source 104 and detector 106 would be reversed from their illustrated positions.

The multilayer plumbing arrangement 240 illustrated in FIGS. 2 and 3 includes a top cap layer 254, a top channel layer 256, an opaque member 102, a bottom channel layer 258, and a bottom cap layer 260. Such layers can be stamped, e.g. die cut, or manufactured by using a laser or water jet, or other machining technique, such as micro machining, etching, and the like. In a currently preferred embodiment 240 that is used to interrogate blood cells, the various layers are typically made from thin polymer films, which are then bonded together to form the multilayer assembly. Exemplary cap layers 254 and 260 may be manufactured from Mylar film that is preferably substantially clear or transparent.

During assembly of a device, bonding may be effected by way of an adhesive applied between one or more layer, or one or more layer may be self-adhesive. It is currently preferred for channel layers 256 and 258 to be manufactured from double-sided tape. One workable tape is made by Adhesive's Research (part no. AR90445). Heat and pressure may also be used, as well as other known bonding techniques. Desirably, the thickness of at least the channel layers 256, 258 is on the order of the characteristic size of particles of interest to promote single-file travel of particles through an interrogation zone. A workable thickness of such layers in currently preferred devices used to interrogate blood cells typically ranges between about 10 microns and about 300 microns.

In certain cases, at least a portion of bottom layer 260 is adapted to form a bottom window 262, through which radiation 118 may be transmitted into an excitation zone. Similarly, top layer 254 includes a portion forming a window 264, through which fluorescence may be transmitted. Therefore, the assembly 240 is arranged to form a window permitting radiation to pass through its thickness. Such window includes window portions 262, 264, certain portions of channels 246 and 248 disposed in the vicinity of orifice 108, and the orifice 108 itself. Radiation can therefore be directed through the thickness of the assembly 240 in the vicinity of the orifice 108.

Emitted fluorescence may be detected by radiation detector 106 of detection structure 55. Presence of a cell may be detected by monitoring a radiological property such as side-scatter, reduction in transmitted radiation due to blockage of aperture 108, or fluorescence. In the event that a cell is detected in the interrogation zone, discrimination structure 57 is operable to distinguish in which population the cell resides. Desirable cells are permitted to pass through the interrogation zone without incident. However, cells in undesired population(s) are killed by killing structure 60, which is discriminately controlled by trigger 75. The resulting collected sample is therefore “purified”, in that the remaining viable cells are all members of a desired population of cells. The “purified” sample may then be manipulated or further interrogated as desired.

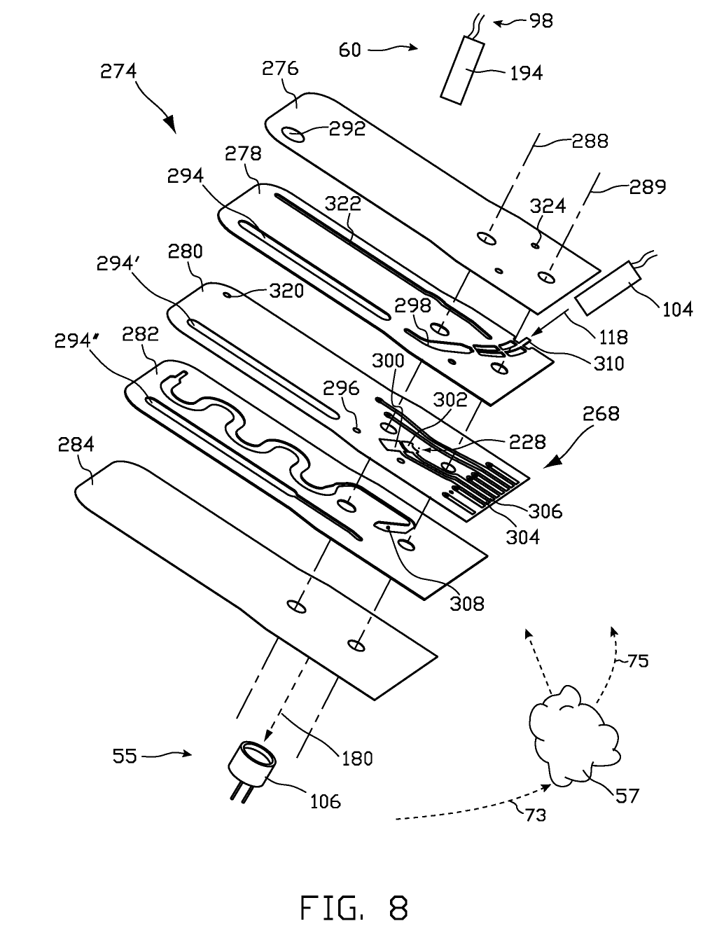

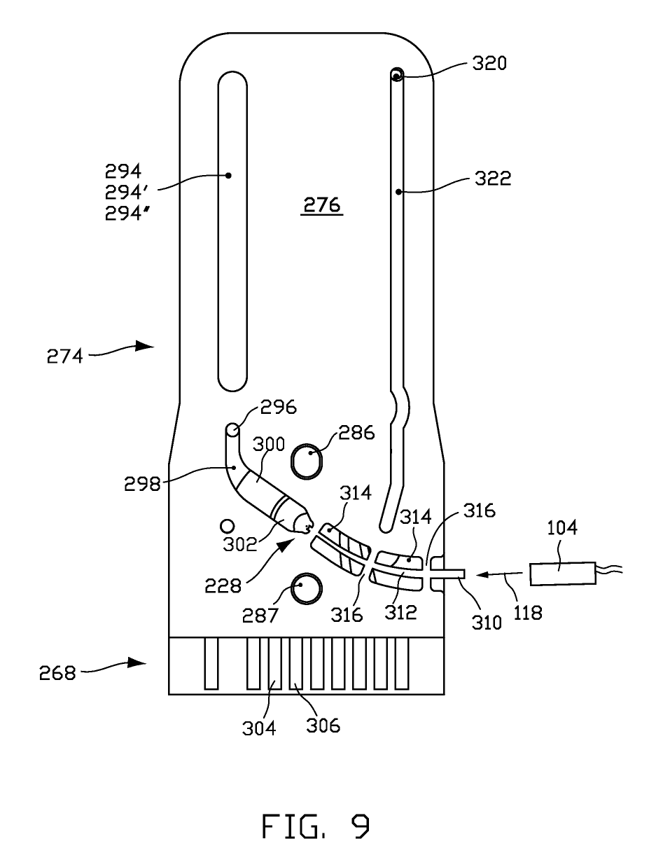

An embodiment structured according to certain principles of the instant invention and permitting either radiological and/or electrically based interrogation of a fluid sample is indicated generally at 274 in FIGS. 8 and 9. Device 274 is particularly adapted as a low-cost, disposable interrogation cartridge for one-time use in combination with a bench-top interrogation platform. As illustrated, device 274 is formed from a plurality of layers, including cap layer 276; channel layer 278, opaque layer 280; channel layer 282, and cap layer 284. Alignment structure, including apertures 286 and 287, facilitates assembly of device 274 by guiding constituent parts along center lines 288 and 289.

In currently preferred embodiments, device 274 is made from, or includes, layers of thin film. Workable films include polymers such as Kapton, Mylar, and the like. Sometimes, one or more layer may be formed from a material, such as injection molded plastic, having an increased thickness to provide enhanced bending stiffness to facilitate handling of the device 274, provide one or more larger known-volume chamber, or for other reasons.

In one exemplary use of device 274, the device is inserted into engagement in an interrogation platform configured to provide the appropriate and desired interrogation capabilities. An interrogation platform typically includes a vacuum source, and one or both of electrical and radiological instrumentation. A fluid sample is placed into sample well 292, where it flows into a chamber defined by chamber-forming voids 294, 294′, and 294″. The fluid is then drawn from channel 294″ through aperture 296 in layer 280, and into channel 298 in layer 278. As illustrated, fluid in channel 298 flows in succession over interrogation electrodes 300 and 302.

With particular reference to FIG. 8, it can be seen that the electrically conductive trace forming interrogation electrode 300 also forms connection electrode 304. Similarly, the conductive trace forming interrogation electrode 302 also forms connection electrode 306. Conductive traces in the illustrated embodiment may be formed on one or both sides of a thin film layer using a well known metallizing procedure, such as photo-masking and etching, vapor deposition, or printing conductive ink. Connection electrodes such as electrode 304 and 306 are configured to permit placing the interrogation electrodes, such as electrodes 300, 302, in circuit with electric interrogation circuitry. A conventional electrical edge connector may conveniently couple with surface-disposed connection electrodes, such as electrodes 304, 306, upon installation of device 274 into an interrogation platform. Such an edge connector may be associated with electrical interrogation circuitry. Therefore, an electrical property of a fluid sample may be interrogated as the sample is drawn through the device 274.

After passing interrogation electrodes 300 and 302, fluid flows downward, through tunnel 228, to channel 308 in layer 282. Additional interrogation electrodes are typically disposed for contact with fluid in channel 308. Such interrogation electrodes may be used, for examples, to detect or interrogate particles moving through tunnel 228 using electrical impedance and the Coulter principle, and/or as one or more event indicator. For example, an event indicator may be used as a start/stop trigger for interrogating a predetermined volume of fluid. Arrival of a fluid wave-front causes a strong change in measured electrical impedance, and indicates the arrival of the wave-front at a first electrode location, which signal may be used to start a test. A subsequent electrode disposed downstream by a known volume may be employed to terminate the test.

As particles move past the tunnel 228, they may also, or alternatively, be interrogated radiologically (e.g. in accordance with Stokes' shift phenomena) at an interrogation zone generally associated with tunnel 228, which is structured to urge particles of interest into substantially single-file transit. As illustrated, stimulation radiation 118 may be introduced from source 104 to a waveguide through pigtail 310. Such an arrangement impinges radiation on an interrogation zone and in a direction substantially transverse to the thickness of the interrogation cartridge.

Impinging radiation in the illustrated transverse direction conveniently reduces the background noise applied to the detector 106, and also reduces need for filters. In alternative construction, an optical fiber may provided as a waveguide structure. It is also operable in certain alternatively structured embodiments to include radiation transmittable windows effective to permit simply impinging excitation radiation in a direction through the thickness of the interrogation cartridge, and to permit collection of Stokes' shift emitted radiation and/or side scatter radiation on the opposite side. One or more band-pass radiation filters would typically be employed in the latter configuration to reduce background noise received at detector 106.

With particular reference to FIG. 9, an exemplary and operable waveguide includes a sidewalk 312 formed by voids 314 formed in a layer. Pillars 316 are provided in the illustrated embodiment 274 to provide stability for sidewalk 312 during assembly of the disposable cartridge 274. The waveguide formed by sidewalk 312 is further exemplary of a focusing light pipe, in which a cross-section of sidewalk 312 is configured to focus radiation transmitted there-through for impingement of focused radiation on an interrogation zone at an increased intensity compared to an intensity of “upstream” radiation, such as radiation received across a transmission interface of the pigtail 310.

Making reference again to FIG. 8, subsequent to filling channel 308, fluid passes through aperture 320, in layer 280, to channel 322 in layer 278. Aperture 324 is provided through layer 276 to permit application of a desired fluid-motive vacuum to channel 322. It has been determined that an O-ring makes an adequate seal in harmony with the top surface of layer 276 at aperture 324 for placing a vacuum source into communication with the cartridge 274 for purpose of causing fluid motion as desired through the cartridge.

As illustrated in FIG. 8, stimulation radiation 118 may be impinged into the interrogation zone associated with aperture 228. Emitted fluorescence may then be detected by radiation detector 106 of detection structure 55. Presence of a cell may be detected by monitoring a radiological property such as side-scatter or fluorescence, and/or by monitoring an electrical property between a pair of electrodes, or between an electrode and a ground reference. In the event that a cell is detected in the interrogation zone, discrimination structure 57 is operable to distinguish in which population the cell resides. Exemplary discrimination structure 57 may distinguish between cells by comparison of real-time detected characteristic values with empirically determined values. Characteristic values that may be compared include the strength of a monitored signal (e.g. peak value) or signal shape over time. Signals that may be monitored include the output from a radiation detector and/or impedance or other electrical property between interrogation electrodes. Desirable cells are permitted to pass through the interrogation zone without incident. However, cells in undesired population(s) are killed (e.g. by laser 194 of killing structure 60, which is discriminately controlled by trigger 75). The resulting collected sample is therefore “purified”, in that the remaining viable cells are all members of a desired population of cells. The “purified” sample may then be manipulated or further interrogated as desired. The sample may be further processed, stored in an on-board chamber, and/or dispensed when desired for further culture or processing of the “purified” sample having viable cells in only the desired population.

An operable plumbing arrangement structured according to certain principles of the instant invention may be manufactured using the following procedure to form an interrogation cartridge: 1. Lay optical fiber (a light pipe) down sandwiched into one of the layers of tape (i.e. laminate). It has been found convenient to use self-adhesive thin film tape, which can be die-cut. The various tape layers will include channels and apertures arranged on assembly to form a fluid conduit extending through the assembly and configured to form an interrogation zone through which particles of interest are urged to move in substantially single-file order. The layer the optical fiber is integrated into will typically have a receiving channel that is cut and sized to receive the fiber. 2. Additional laminate layers, or adhesive, may be added to keep the fiber in position. 3. The sub-assembly may then be sent to a laser drilling house to drill the cell sensing zone (CSZ) hole, or aperture, through the opaque layer. The hole will desirably be drilled relative to the location of the fiber (i.e., just off the end of the tip of the fiber). 4. The assembly is then typically finished when the final laminate cap layers (typically clear Mylar layers) are added. Sometimes, a stiffening substrate may be included to facilitate handling of the interrogation cartridge.

Certain components that are operable to construct an apparatus according to certain principles of the instant invention are commercially available. For example, one operable source of radiation 104 includes a red diode laser available under part number VPSL-0639-035-x-5-B, from Blue Sky Research, having a place of business located at 1537 Centre Point Drive, Milpitas, Calif. 95035. Filter elements 188, 190 are available from Omega Optical, having a place of business located at 21 Omega Dr., Delta Campus, Brattleboro, Vt. 05301. Preferred filters include part numbers, 655LP or 660NB5 (Bandpass filter), and 640ASP (shortpass filter). An operable radiation detector 106 includes a photomultiplier tube available from the Hamamatsu Corporation, having a place of business located at 360 Foothill Rd., Bridgewater, N.J. 08807, under part number H5784-01. A workable killing laser 194 is available under part Number IQ 1C16 from Power Technology. Molecular Probes (a division of Invitrogen Corporation, www.probes.invitrogen.com) supplies a plurality dyes that are suitable for use in tagging certain particles of interest for interrogation using embodiments structured according to the instant invention. In particular, AlexaFluor 647, AlexaFluor 700, and APC-AlexaFluor 750 find application to interrogation of blood cells. These dyes are also commonly used in flow cytometric applications and have specific excitation and emission characteristics. Each dye can be easily conjugated to antibodies for labeling, or tagging, different cell types. An operable fiber optic cable for forming a waveguide is available under part No. BK-0100-07 from Thor Labs, having a web site address of http://www.thorlabs.com. One useful fiber diameter is about 0.010″.

In one method for using the invention, particles (e.g. blood cells) of interest are mixed with a commercially available or custom manufactured antibody-bound fluorescently labeled molecules (i.e., obtained from Invitrogen Corporation, Carlsbad, Calif.). The mixture is then incubated for a brief period of time (approximately 5 to 15 minutes) at a temperature typically between about room temperature and abut 39 degrees Celsius. For preparation of white blood cells for interrogation, a small amount of fluorescent dye (e.g. 10 microliters) is added to about 10 microliters of whole blood, vortexed and then incubated for about 15 minutes at room temperature in the dark. A lysing agent is then added to lyse the red blood cells. Once added, the mixture is again vortexed and then allowed to incubate for another 15 minutes (in the dark).

Fluorescent markers bind to target cells (or other biological particles of interest) in the sample during the incubation period. The particles suspended in solution are then passed through the orifice detection zone from one (supply) reservoir to another (holding) reservoir, typically by applying either an external vacuum source to pull the sample through or an external positive gas source to push the sample through. Fluorescently labeled particles are excited with primary radiation (light) as they traverse the opaque member (e.g. through the interrogation orifice of a device such as 274 in FIGS. 8 and 9) which causes fluorescence and subsequent emission of light having a secondary wavelength (which is released into the opposite or detector side of the opaque member). Presence of particles in the interrogation zone may be detected optically, radiologically, or electrically with suitable detection structure. Discrimination structure (e.g. including a radiation detector to monitor for Stokes' shift phenomena) is used to distinguish in which population a given particle resides. Particles residing in undesired populations are killed by the killing structure. Living (and dead) particles flow away from the interrogation and killing zone to the holding reservoir or storage containment area. The thus “purified” sample may subsequently be dispensed into a container for further manipulation and interrogation.

While the invention has been described in particular with reference to certain illustrated embodiments, such is not intended to limit the scope of the invention. The present invention may be embodied in other specific forms without departing from its spirit or essential characteristics. The described embodiments are to be considered as generally illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
alignment structure configured and arranged to urge biological particles, which are carried in a fluid, toward substantially single-file travel through an interrogation zone;
detection structure operable to detect the presence of a first biological particle in said interrogation zone;
discrimination structure operable to distinguish said first biological particle as either residing inside a defined population of particles, or not;
killing structure configured and arranged substantially discriminately to kill a selected biological particle in a killing zone that is associated with said interrogation zone; and
a trigger operable to actuate said killing structure responsive to input received from one or both of said detection structure and said discrimination structure.

2. The apparatus according to claim 1, wherein:
said alignment structure comprises a fluid sheath.

3. The apparatus according to claim 1, wherein:
said alignment structure comprises a capillary device.

4. The apparatus according to claim 1, wherein:
said alignment structure comprises a fluid-carrying channel formed in a thin film layer.

5. The apparatus according to claim 1, wherein:
said detection structure comprises a plurality of electrodes disposed in operable association with an orifice effective to permit detecting the presence of a particle in said interrogation zone by way of the Coulter principle.

6. The apparatus according to claim 1, wherein:
said detection structure comprises:
a radiation source disposed to impinge radiation comprising substantially a first frequency into said interrogation zone; and
a radiation detector disposed to detect a Stokes' shift in said substantially first frequency.

7. The apparatus according to claim 1, wherein:
said detection structure comprises:
a radiation source disposed to impinge radiation comprising substantially a first frequency into said interrogation zone; and
a radiation detector disposed to detect side-scatter of said radiation.

8. The apparatus according to claim 1, wherein:
said trigger is adapted to operate said killing structure in the case when a detected biological particle is both:
present in said killing zone; and
resides inside said defined population of particles.

9. The apparatus according to claim 1, wherein:
said trigger is adapted to operate said killing structure in the case when a detected biological particle is both:
present in said killing zone; and
resides outside said defined population of particles.

10. The apparatus according to claim 1, wherein:
said killing zone is disposed as a sub-portion of said interrogation zone.

11. The apparatus according to claim 1, wherein:
said killing zone is disposed downstream of said interrogation zone by a known time-of-flight for a biological particle to be killed.

12. The apparatus according to claim 1, wherein:
said killing zone is disposed downstream of said detection structure by a known time-of-flight for a biological particle to be killed.

13. The apparatus according to claim 1, wherein:
said killing structure comprises a radiation source having sufficient discharged energy density to permit exposing a biological particle, during the time said biological particle is passing through said killing zone, to at least that quantity of energy sufficient to kill said biological particle.

14. The apparatus according to claim 1, wherein:
said killing structure comprises a laser.

15. The apparatus according to claim 1, wherein:
said interrogation zone is carried on a disposable device that is adapted for one-time-use.

16. The apparatus according to claim 15, wherein:
said disposable device is embodied as a microfluidic cartridge.

17. A microfluidic apparatus, comprising:
alignment structure configured and arranged to urge biological particles, which are carried in a fluid, toward substantially single-file travel through an interrogation zone;
detection structure operable to detect the presence of a first biological particle in said interrogation zone using electrical impedance in accordance with the Coulter principle;
discrimination structure operable to distinguish said first biological particle as either residing inside a defined population of particles, or not;
killing structure configured and arranged substantially discriminately to kill a selected biological particle in a killing zone that is associated with said interrogation zone; and
a trigger operable to actuate said killing structure responsive to input received from both of said detection structure and said discrimination structure.

18. A method to sort biological particles, comprising the steps of:
providing a microfluidic device comprising:
alignment structure configured and arranged to urge biological particles, which are carried in a fluid, toward substantially single-file travel through an interrogation zone;
detection structure operable to detect the presence of a first biological particle in said interrogation zone;
discrimination structure operable to distinguish said first biological particle as either residing inside a defined population of particles, or not; and
killing structure configured and arranged substantially discriminately to kill a selected biological particle in a killing zone that is associated with said interrogation zone;
introducing a fluid sample, comprising biological particles carried by a dilutant fluid medium, for flow of said sample past said alignment structure; and
operating a trigger to actuate said killing structure effective to kill a sub-population of biological particles responsive to input received from one or both of said detection structure and said discrimination structure as said sample flows through said device.

19. The method according to claim 18, wherein:
said sub-population is defined by a common characteristic that is directly detected by said discrimination structure.

20. The method according to claim 18, wherein:
said sub-population is defined by a common characteristic that is not directly detected by said discrimination structure.
